(19) United States
(12) Patent Application Publication
(10) Pub. No.: US 2026/0083417 A1
(43) Pub. Date: Mar. 26, 2026

(54) Display Method in X-Ray Imaging Apparatus and X-Ray Imaging Apparatus

(71) Applicant: SHIMADZU CORPORATION, Kyoto-shi (JP)

(72) Inventor: Ken SHIROTA, Kyoto-shi, (JP)

(21) Appl. No.: 19/139,695

(22) PCT Filed: Sep. 8, 2023
(86) PCT No.: PCT/JP2023/032917

§ 371 Date: Jun. 16, 2025

(30) Foreign Application Priority Data
Dec. 16, 2022 (JP) 2022-201436

(51) Int. Cl. A61B 6/46; A61B 6/00; G06T 11/00

(52) U.S. Cl. CPC A61B 6/465; A61B 6/54; G06T 11/00; G06T 2200/24; G06T 2210/41

(57) ABSTRACT

A display method in an X-ray imaging apparatus (100) includes a step of simultaneously and individually executing, on a single control device (40), X-ray image generation software (61) being a product of a first manufacturer, and imaging condition setting software (62) being a product of a second manufacturer to form an X-ray image display screen (71) and an imaging condition setting screen (72), and a step of displaying the X-ray image display screen and the imaging condition setting screen side by side in different areas of a display (70).

### TECHNICAL FIELD

The present invention relates to a display method in an X-ray imaging apparatus and an X-ray imaging apparatus.

### BACKGROUND ART

Conventionally, an X-ray imaging apparatus including an X-ray source, an X-ray detector, and a display is known. Such an X-ray imaging apparatus is disclosed in Japanese Patent Laid-Open No. 2018-033552, for example.

Japanese Patent Laid-Open No. 2018-033552 discloses that an X-ray diagnostic apparatus (X-ray imaging apparatus) includes an X-ray tube (X-ray source), an X-ray detector, and a console (display) including a display. Japanese Patent Laid-Open No. 2018-033552 discloses that the X-ray detector converts information about X-rays that have passed through a subject into image data and transmits it to the console in an operations room.

Japanese Patent Laid-Open No. 2018-033552 discloses that the console is a single console in which a system for setting and managing imaging conditions and collecting image data is integrated with a system for processing and displaying an image. Japanese Patent Laid-Open No. 2018-033552 discloses that the console displays the imaging conditions on the display and receives settings of the imaging conditions from an operator. In addition, Japanese Patent Laid-Open No. 2018-033552 discloses that the console collects captured image data and performs preset image processing on the collected image data, changes image processing based on an operation from a mouse, a keyboard, or the like, and displays an image on the display.

### PRIOR ART

### Patent Document

Patent Document 1: Japanese Patent Laid-Open No. 2018-033552

### SUMMARY OF THE INVENTION

### Problems to be Solved by the Invention

Although not clearly described in Japanese Patent Laid-Open No. 2018-033552, in the X-ray diagnostic apparatus disclosed in Japanese Patent Laid-Open No. 2018-033552, the system for setting the imaging conditions and the system including the X-ray detector for processing and displaying an image, for example, which are integrated, are conceivably manufactured by the same manufacturer. In addition, although not clearly described in Japanese Patent Laid-Open No. 2018-033552, software used to set the imaging conditions and software used to process and display an image are also conceivably integrated by integrating these systems. Therefore, in the X-ray diagnostic apparatus disclosed in Japanese Patent Laid-Open No. 2018-033552, a screen for processing and displaying an image and a screen for setting the imaging conditions can be conceivably displayed on one console.

On the other hand, although not disclosed in Japanese Patent Laid-Open No. 2018-033552, an X-ray detector manufactured by a different manufacturer may be used in combination in an X-ray diagnostic apparatus (X-ray imaging apparatus). In other words, in an X-ray imaging apparatus, a system for setting imaging conditions and a system including an X-ray detector for processing and displaying an image, both of which are manufactured by different manufacturers, may be used. In such a case, these systems are not integrated, and thus software used to set the imaging conditions (imaging condition setting software) and software used to process and display an image (X-ray image generation software) are not integrated. Therefore, it is difficult to display a screen for processing and displaying an image and a screen for setting the imaging conditions on one console. Thus, the screen displayed by the software used to process and display an image and the screen displayed by the software used to set the imaging conditions are displayed on separate display devices. Therefore, it is desired to achieve an environment in which the independently provided X-ray image generation software and imaging condition setting software can be simultaneously and individually executed by a flexible combination without restrictions imposed by the manufacturer of the X-ray imaging apparatus and the manufacturer of the X-ray detector.

The present invention is intended to solve at least one of the above problems. The present invention aims to provide a display method in an X-ray imaging apparatus and an X-ray imaging apparatus in which an environment in which independently provided x-ray image generation software and imaging condition setting software can be simultaneously and individually executed can be achieved by a flexible combination without restrictions imposed by a manufacturer of the X-ray imaging apparatus and a manufacturer of an X-ray detector.

### Means for Solving the Problems

A display method in an X-ray imaging apparatus according to a first aspect of the present invention includes a screen formation step of simultaneously and individually executing, on a single control device, X-ray image generation software operable to form an X-ray image based on a detection signal output from an X-ray detector and form an X-ray image display screen, the X-ray image generation software being a product of a first manufacturer, and imaging condition setting software independent of the X-ray image generation software, the imaging condition setting software being operable to set an imaging condition of the X-ray image and form an imaging condition setting screen, the imaging condition setting software being a product of a second manufacturer different from the first manufacturer to form the X-ray image display screen based on the X-ray image generation software and the imaging condition setting screen based on the imaging condition setting software, and a screen display step of displaying the X-ray image display screen and the imaging condition setting screen side by side in different areas of a display.

An X-ray imaging apparatus according to a second aspect of the present invention includes an X-ray source to radiate X-rays to a subject, an X-ray detector to detect the X-rays radiated from the X-ray source, a display, and a single controller configured or programmed to simultaneously and individually execute X-ray image generation software operable to form an X-ray image based on a detection signal output from the X-ray detector and form an X-ray image display screen, the X-ray image generation software being a product of a first manufacturer, and imaging condition setting software independent of the X-ray image generation software, the imaging condition setting software being operable to set an imaging condition of the X-ray image and form an imaging condition setting screen, the imaging condition setting software being a product of a second manufacturer different from the first manufacturer to form the X-ray image display screen based on the X-ray image generation software and the imaging condition setting screen based on the imaging condition setting software, and display the X-ray image display screen based on the X-ray image generation software and the imaging condition setting screen based on the imaging condition setting software side by side in different areas of the display.

An X-ray imaging apparatus according to a third aspect of the present invention includes an X-ray source to radiate X-rays to a subject, an X-ray detector to detect the X-rays radiated from the X-ray source, a display, and a controller configured or programmed to display an X-ray image display screen based on X-ray image generation software operable to form an X-ray image based on a detection signal output from the X-ray detector and form the X-ray image display screen, and an imaging condition setting screen based on imaging condition setting software independent of the X-ray image generation software, the imaging condition setting software being operable to set an imaging condition of the X-ray image and form the imaging condition setting screen side by side in different areas of the display. The controller is configured or programmed to perform a control such that a total number of pixels of the X-ray image display screen is smaller than a total number of pixels of an entirety of the display when the X-ray image display screen is formed using the X-ray image generation software, and perform a control such that a total number of pixels of the imaging condition setting screen is equal to or less than a number of pixels obtained by subtracting the total number of pixels of the X-ray image display screen from the total number of pixels of the entirety of the display when the imaging condition setting screen is formed using the imaging condition setting software.

### Advantageous Effect of the Invention

As described above, the display method in an X-ray imaging apparatus according to the first aspect of the present invention includes the screen formation step of simultaneously and individually executing, on the single control device, the X-ray image generation software being the product of the first manufacturer, and the imaging condition setting software independent of the X-ray image generation software and being the product of the second manufacturer to form the X-ray image display screen based on the X-ray image generation software and the imaging condition setting screen based on the imaging condition setting software, and the screen display step of displaying the X-ray image display screen and the imaging condition setting screen side by side in the different areas of the display. Accordingly, the X-ray image generation software and the imaging condition setting software, which are made by different manufacturers, are simultaneously and individually executed on the single control device such that the X-ray image display screen and the imaging condition setting screen can be formed and displayed in the different areas of the display. Therefore, an environment in which the independently provided X-ray image generation software and imaging condition setting software can be executed simultaneously and individually can be achieved by a flexible combination without restrictions imposed by the manufacturer of the X-ray imaging apparatus and the manufacturer of the X-ray detector.

In the X-ray imaging apparatus according to the second aspect, as described above, the controller is a single controller, and is configured or programmed to simultaneously and individually execute the X-ray image generation software being the product of the first manufacturer, and the imaging condition setting software independent of the X-ray image generation software and being the product of the second manufacturer to form the X-ray image display screen based on the X-ray image generation software and the imaging condition setting screen based on the imaging condition setting software, and display the X-ray image display screen and the imaging condition setting screen side by side in the different areas of the display. Accordingly, the X-ray image generation software and the imaging condition setting software, which are made by different manufacturers, are simultaneously and individually executed on the single control device such that the X-ray image display screen and the imaging condition setting screen can be formed and displayed in the different areas of the display. Therefore, an environment in which the independently provided X-ray image generation software and imaging condition setting software can be simultaneously and individually executed can be achieved by a flexible combination without restrictions imposed by the manufacturer of the X-ray imaging apparatus and the manufacturer of the X-ray detector.

In the X-ray imaging apparatus according to the third aspect, as described above, the controller is configured or programmed to display the X-ray image display screen based on the X-ray image generation software and the imaging condition setting screen based on the imaging condition setting software independent of the X-ray image generation software side by side in the different areas of the display, to perform a control such that the total number of pixels of the X-ray image display screen is smaller than the total number of pixels of the entirety of the display when the X-ray image display screen is formed using the X-ray image generation software, and to perform a control such that the total number of pixels of the imaging condition setting screen is equal to or less than the number of pixels obtained by subtracting the total number of pixels of the X-ray image display screen from the total number of pixels of the entirety of the display when the imaging condition setting screen is formed using the imaging condition setting software. The X-ray image display screen based on the X-ray image generation software and the imaging condition setting screen based on the imaging condition setting software are displayed side by side in the different areas of the display such that an environment in which the independently provided X-ray image generation software and imaging condition setting software can be simultaneously and individually executed can be achieved by a flexible combination without restrictions imposed by the manufacturer of the X-ray imaging apparatus and the manufacturer of the X-ray detector. In addition, the X-ray image generation software forms the X-ray image display screen such that the total number of pixels of the X-ray image display screen is smaller than the total number of pixels of the entirety of the display in the X-ray imaging apparatus, the imaging condition setting software forms the imaging condition setting screen such that the total number of pixels of the imaging condition setting screen is equal to or less than the number of pixels obtained by subtracting the total number of pixels of the X-ray image display screen from the total number of pixels of the entirety of the display, and the X-ray image display screen and the imaging condition setting screen are displayed side by side in the different areas of the display. Thus, the imaging condition setting screen can be displayed in an area of the single display excluding the area in which the X-ray image display screen is displayed, and thus both the X-ray image display screen based on the X-ray image generation software and the imaging condition setting screen based on the imaging condition setting software can be displayed on the display. Therefore, when the X-ray image generation software and the imaging condition setting software are not integrated, the X-ray image display screen and the imaging condition setting screen are displayed on the single display such that the complexity of the configuration of the display and an increase in the number of displays can be reduced or prevented.

### BRIEF DESCRIPTION OF THE DRAWINGS

FIG. 1 is a schematic view of an X-ray imaging apparatus according to an embodiment.

FIG. 2 is a block diagram showing an outline of the X-ray imaging apparatus according to the embodiment.

FIG. 3 is a schematic view of an X-ray image display screen and an imaging condition setting screen.

FIG. 4 is a flowchart for illustrating a sequence of display processing of the X-ray image display screen and the imaging condition setting screen.

### MODES FOR CARRYING OUT THE INVENTION

An embodiment embodying the present invention is hereinafter described on the basis of the drawings.

### Configuration of X-Ray Imaging Apparatus

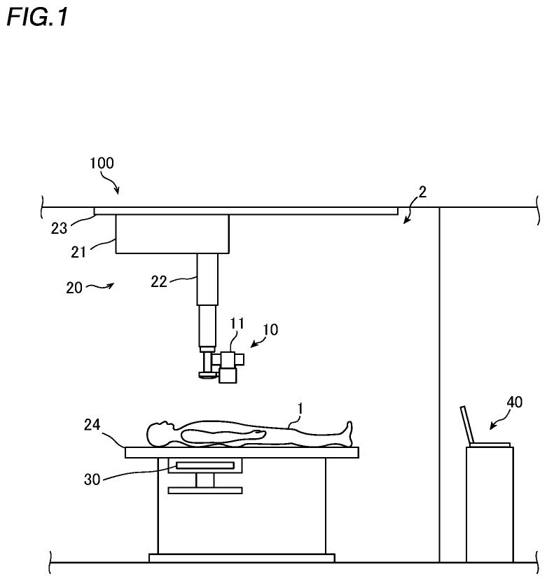

The configuration of an X-ray imaging apparatus 100 according to the embodiment of the present invention is now described with reference to FIGS. 1 to 3.

As shown in FIG. 1, the X-ray imaging apparatus 100 includes an X-ray irradiator 10, a movement mechanism 20, an X-ray detector 30, and a control device 40.

The X-ray imaging apparatus 100 is a medical X-ray imaging apparatus configured to capture an X-ray image of a subject 1 (patient) that is a target to be imaged. The X-ray imaging apparatus 100 is installed in an examination room 2 in which medical procedures on the subject 1 are performed.

The X-ray irradiator 10 includes an X-ray source 11. The X-ray source 11 is configured to irradiate the subject 1 with X-rays.

The movement mechanism 20 is configured to hold the X-ray irradiator 10 such that the X-ray irradiator 10 is movable. The movement mechanism 20 includes an overhead suspension 21 and a supporting column 22. The movement mechanism 20 is supported by a rail 23 provided on the ceiling of an imaging room. The overhead suspension 21 is movable in a horizontal direction by the rail 23. The overhead suspension 21 is configured to support the supporting column 22. The supporting column 22 is configured to support the X-ray irradiator 10. The supporting column 22 is extendable and retractable in a vertical direction. The X-ray irradiator 10 is movable in the vertical direction by the supporting column 22.

The X-ray detector 30 is configured to detect X-rays radiated from the X-ray irradiator 10. The X-ray detector 30 includes, for example, a digital radiography (DR) flat panel detector (FPD). The X-ray detector 30 is provided on a table 24 for imaging the subject 1 in a posture (lying position) in which the subject 1 is lying. During X-ray imaging, the X-ray irradiator 10 is disposed at a position vertically facing the X-ray detector 30 on the table 24. The X-ray imaging apparatus 100 images the subject 1 lying on the table 24 between the X-ray source 11 and the X-ray detector 30 that vertically face each other.

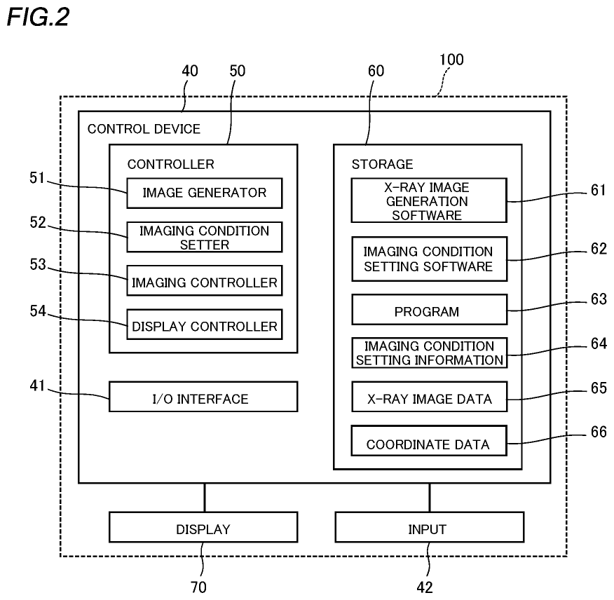

As shown in FIG. 2, the control device 40 includes a personal computer (PC), for example. The control device 40 is a single device. The control device 40 includes a controller 50, a storage 60, and an I/O interface 41. The control device 40 is connected to a display 70 and an input 42.

The controller 50 includes a processor including a central processing unit (CPU), a read-only memory (ROM), and a random access memory (RAM), a graphics processing unit (GPU), or a field-programmable gate array (FPGA) configured for image processing, for example. The controller 50 is configured as a single controller.

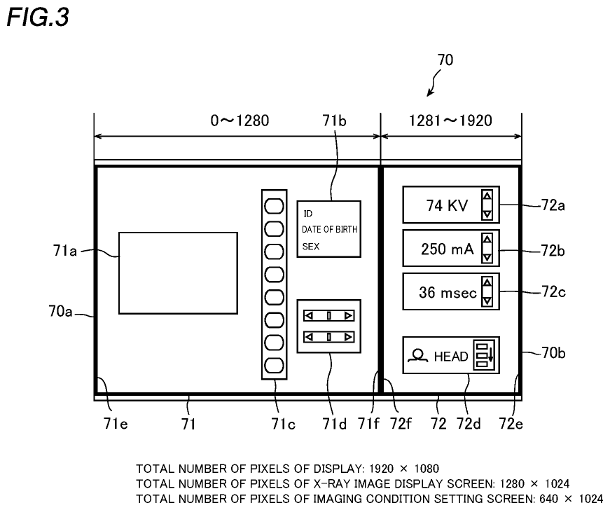

The controller 50 executes X-ray image generation software 61 (application software) stored in the storage 60 to generate an X-ray image based on a detection signal output from the X-ray detector 30. The controller 50 also executes the X-ray image generation software 61 to perform a control to form an X-ray image display screen 71 (see FIG. 3) for displaying the generated X-ray image.

The controller 50 executes imaging condition setting software 62 (application software) stored in the storage 60 to set imaging conditions for an X-ray image. The controller 50 also executes the imaging condition setting software 62 to set the imaging conditions and to perform a control to form an imaging condition setting screen 72 (see FIG. 3) for displaying the set imaging conditions.

The controller 50 performs a control to display the X-ray image display screen 71 (see FIG. 3) based on the X-ray image generation software 61 and the imaging condition setting screen 72 (see FIG. 3) based on the imaging condition setting software 62 side by side in different areas of the display 70.

The controller 50 executes the X-ray image generation software 61 and the imaging condition setting software 62 simultaneously and individually to perform a control to form the X-ray image display screen 71 based on the X-ray image generation software 61 and the imaging condition setting screen 72 based on the imaging condition setting software 62, and display, on the display 70, the X-ray image display screen 71 based on the X-ray image generation software 61 and the imaging condition setting screen 72 based on the imaging condition setting software 62.

The X-ray image generation software 61 is software for generating the X-ray image based on the detection signal output from the X-ray detector 30. The X-ray image generation software 61 is also software for forming the X-ray image display screen 71 (see FIG. 3) that is displayed on the display 70. The X-ray image generation software 61 is a product of a first manufacturer, for example.

The imaging condition setting software 62 is software for setting the imaging conditions for an X-ray image. The imaging condition setting software 62 is also software for forming the imaging condition setting screen 72 (see FIG. 3) that is displayed on the display 70. The imaging condition setting software 62 is provided independently of the X-ray image generation software 61. The imaging condition setting software 62 is a product of a second manufacturer different from the first manufacturer, for example.

The storage 60 includes a volatile storage device and a non-volatile storage device. The storage 60 stores the X-ray image generation software 61, the imaging condition setting software 62, a program 63, setting information 64 of the X-ray imaging conditions of the X-ray imaging apparatus 100, acquired X-ray image data 65, coordinate data 66 of the display positions of the X-ray image display screen 71 and the imaging condition setting screen 72 written in a setting file (not shown), etc.

The I/O interface 41 includes various interfaces to input and output signals to and from the control device 40. The I/O interface 41 is connected to the display 70 and the input 42. The display 70 is, for example, a liquid crystal display. The input 42 is, for example, a keyboard and a mouse. The controller 50 acquires a detection signal (image signal) from the X-ray detector 30 via the I/O interface 41. The controller 50 also controls the operation of the X-ray irradiator 10 to start or stop imaging via the I/O interface 41.

As shown in FIG. 3, as an example, the display 70 includes the X-ray image display screen 71 and the imaging condition setting screen 72. On the display 70, the X-ray image display screen 71 and the imaging condition setting screen 72 are displayed side by side in the different areas. On the display 70, the X-ray image display screen 71 and the imaging condition setting screen 72 are displayed in this order from the left side of the display 70 when a user looks at the display 70. For convenience of explanation, the X-ray image display screen 71 and the imaging condition setting screen 72 are indicated by thick lines.

The X-ray image display screen 71 includes an X-ray image display area 71a, a subject information display area 71b, a toolbar 71c, and an image processing operation area 71d. In the X-ray image display area 71a, an X-ray image generated by an image generator 51 (see FIG. 2) described below is displayed. In the subject information display area 71b, subject information about the subject 1 appearing on the displayed X-ray image is displayed. The subject information includes a subject ID, the date of birth of the subject, the sex of the subject, etc., for example. The displayed X-ray image can be enlarged or reduced by performing an input operation on the toolbar 71c, for example. Image processing such as gradation processing can be performed by performing an input operation on the image processing operation area 71d, for example.

The imaging condition setting screen 72 displays information about the imaging conditions for X-ray imaging that can be set by an imaging condition setter 52 (see FIG. 2) described below. As an example, the imaging condition setting screen 72 displays information 72a about a tube voltage in an X-ray tube (not shown), information 72b about a tube current, and information 72c about an X-ray irradiation time, information 72d about an imaging region of the subject 1, etc. that are set by the user or are initially set. Furthermore, the imaging condition setter 52 is configured to allow the user to change, on the imaging condition setting screen 72, settings of the tube voltage, tube current, and X-ray irradiation time in the X-ray tube, the imaging region of the subject 1, etc. by an input operation.

### Processing of Controller

Processing performed by the controller 50 is now described with reference to FIG. 2. The controller 50 including hardware such as a CPU includes the image generator 51, the imaging condition setter 52, an imaging controller 53, and a display controller 54 as functional blocks of software (application software) and the program 63. The controller 50 functions as the image generator 51 by executing the X-ray image generation software 61 stored in the storage 60. The controller 50 also functions as the imaging condition setter 52 by executing the imaging condition setting software 62 stored in the storage 60. The controller 50 also functions as the display controller 54 and the imaging controller 53 by executing the program 63 stored in the storage 60.

The image generator 51 is configured to generate an X-ray image based on the X-rays detected by the X-ray detector 30 (see FIG. 1). The image generator 51 is also configured to form the X-ray image display screen 71 (see FIG. 3). The image generator 51 is also configured to display the generated X-ray image on the X-ray image display screen 71. The image generator 51 is also configured to perform image processing such as gradation processing on the X-ray image displayed on the X-ray image display screen 71 based on an input instruction by the user for an operation image.

The image generator 51 is configured to, when the X-ray image generation software 61 is executed to form the X-ray image display screen 71 (see FIG. 3), perform a control such that the total number of pixels of the X-ray image display screen 71 is smaller than the total number of pixels of the entirety of the display 70. Specifically, the image generator 51 is configured to, when the X-ray image generation software 61 is executed to form the X-ray image display screen 71, perform a control such that the total number of pixels of the X-ray image display screen 71 is a total number of pixels pre-selected from a plurality of types of total numbers of pixels set in advance such that the X-ray image display screen 71 is displayable.

The X-ray image generation software 61 that is generally used is configured such that the X-ray image display screen 71 can be displayed in accordance with the plurality of types of total numbers of pixels in order for the X-ray image display screen 71 to be displayed full-screen on each of a plurality of displays 70 having different total numbers of pixels. That is, the X-ray image generation software 61 is configured such that the X-ray image display screen 71 can be displayed full-screen on, for example, a display 70 having a total number of pixels of 1920 horizontal pixels×1080 vertical pixels, a display 70 having a total number of pixels of 1280 horizontal pixels×1024 vertical pixels, a display 70 having a total number of pixels of 1600 horizontal pixels×1200 vertical pixels, and a display 70 having a total number of pixels of 800 horizontal pixels×600 vertical pixels. The X-ray image generation software 61 is configured such that the X-ray image display screen 71 is displayed with an appropriate total number of pixels for full-screen display corresponding to the above-described display 70 given as an example, and when the display 70 is changed to a display 70 having a different total number of pixels, switching to an appropriate total number of pixels for full-screen display corresponding to the changed display 70 is performed and the X-ray image display screen 71 is displayed.

In the present invention, while using the above-described X-ray image generation software 61, the image generator 51 does not form the X-ray image display screen 71 by full-screen display on the display 70, but forms the X-ray image display screen 71 on the display 70 with a number of pixels smaller than the total number of pixels of the entirety of the display 70. At this time, the total number of pixels of the formed X-ray image display screen 71 is a total number of pixels pre-selected from the plurality of types of total numbers of pixels set in advance such that the X-ray image display screen 71 is displayable.

As an example, as shown in FIG. 3, the X-ray image generation software 61 is configured such that the X-ray image display screen 71 can be displayed full-screen on, for example, the display 70 having a total number of pixels of 1920 horizontal pixels×1080 vertical pixels, the display 70 having a total number of pixels of 1280 horizontal pixels×1024 vertical pixels, the display 70 having a total number of pixels of 1600 horizontal pixels×1200 vertical pixels, and the display 70 having a total number of pixels of 800 horizontal pixels×600 vertical pixels, and when the display 70 has a total number of pixels of 1920 horizontal pixels×1080 vertical pixels, the image generator 51 forms the X-ray image display screen 71 on the display 70 with a total number of pixels of 1280 horizontal pixels×1024 vertical pixels pre-selected from the plurality of types of total numbers of pixels set in advance. In this embodiment, the total number of pixels of the X-ray image display screen 71 formed on the display 70 is selected when the X-ray imaging apparatus 100 is installed.

As shown in FIG. 2, the imaging condition setter 52 is configured to form the imaging condition setting screen 72 (see FIG. 3). The imaging condition setter 52 is also configured to set the tube voltage, tube current, and X-ray irradiation time in the X-ray tube (not shown), the imaging region of the subject 1, etc. based on an input instruction from the user. The imaging condition setter 52 is also configured to display, on the imaging condition setting screen 72, the information 72a about the tube voltage in the X-ray tube, the information 72b about the tube current, and the information 72c about the X-ray irradiation time, the information 72d about the imaging region of the subject 1, etc. that are set by the user or are initially set.

The imaging condition setter 52 is configured to, when the imaging condition setting software 62 is executed to form the imaging condition setting screen 72, perform a control such that the total number of pixels of the imaging condition setting screen 72 is equal to or less than the number of pixels obtained by subtracting the total number of pixels of the X-ray image display screen 71 from the total number of pixels of the entirety of the display 70. Specifically, the imaging condition setter 52 is configured to, when the imaging condition setting software 62 is executed to form the imaging condition setting screen 72, perform a control such that the total number of pixels of the imaging condition setting screen 72 is a total number of pixels pre-selected from a plurality of types of total numbers of pixels set in advance such that the imaging condition setting screen 72 is displayable in an area of the display 70 in which the X-ray image display screen 71 is not displayed.

The imaging condition setting software 62 is configured such that the imaging condition setting screen 72 can be formed with the plurality of types of total numbers of pixels so as to correspond to a plurality of X-ray image display screens 71 having different total numbers of pixels formed on the plurality of displays 70 having different total numbers of pixels. The imaging condition setter 52 forms the imaging condition setting screen 72 on the display 70 with the number of pixels equal to or less than the number of pixels obtained by subtracting the total number of pixels of the X-ray image display screen 71 from the total number of pixels of the entirety of the display 70. At this time, the total number of pixels of the formed imaging condition setting screen 72 is a total number of pixels pre-selected from the plurality of types of total numbers of pixels set in advance such that the imaging condition setting screen 72 is displayable in the area in which the X-ray image display screen 71 is not displayed.

As an example, as shown in FIG. 3, when the display 70 has a total number of pixels of 1920 horizontal pixels×1080 vertical pixels, and the image generator 51 forms the X-ray image display screen 71 with a pre-selected total number of pixels of 1280 horizontal pixels×1024 vertical pixels, the imaging condition setter 52 forms the imaging condition setting screen 72 on the display 70 with a total number of pixels of 640 horizontal pixels×1024 vertical pixels pre-selected from the plurality of types of total numbers of pixels set in advance. In this embodiment, the total number of pixels of the imaging condition setting screen 72 formed on the display 70 is selected when the X-ray imaging apparatus 100 is installed.

As shown in FIG. 2, the imaging controller 53 performs a control to radiate X-rays from the X-ray source 11 and a control to stop the X-ray radiation, based on the imaging conditions set by the imaging condition setter 52.

The display controller 54 is configured or programmed to display the X-ray image display screen 71 based on the X-ray image generation software 61 and the imaging condition setting screen 72 based on the imaging condition setting software 62 side by side in the different areas of the display 70.

The display controller 54 is configured or programmed to display the X-ray image display screen 71 formed by the image generator 51 at a fixed position and with a fixed size on the display 70 in a state in which the X-ray image display screen 71 cannot be enlarged or reduced. The display controller 54 is also configured or programmed to display the imaging condition setting screen 72 formed by the imaging condition setter 52 at a fixed position and with a fixed size on the display 70 in a state in which the imaging condition setting screen 72 cannot be enlarged or reduced.

As an example, as shown in FIG. 3, the display controller 54 is configured or programmed to display the X-ray image display screen 71 with a total number of pixels of 1280 horizontal pixels×1024 vertical pixels at a fixed position and with a fixed size on the left side of the display 70 in a state in which the X-ray image display screen 71 cannot be enlarged or reduced. The display controller 54 is also configured or programmed to display the imaging condition setting screen 72 with a total number of pixels of 640 horizontal pixels×1024 vertical pixels at a fixed position and with a fixed size on the right side of the display 70 in a state in which the imaging condition setting screen 72 cannot be enlarged or reduced.

That is, the display controller 54 is configured or programmed to prohibit the user from changing the display size and display position of each of the X-ray image display screen 71 and the imaging condition setting screen 72 and displaying only one of the X-ray image display screen 71 and the imaging condition setting screen 72, during activation of the control device 40. Thus, the X-ray imaging apparatus 100 is configured to automatically display the X-ray image display screen 71 and the imaging condition setting screen 72 at fixed positions and with fixed sizes on the display 70 in a state in which the X-ray image display screen 71 and the imaging condition setting screen 72 cannot be enlarged or reduced, from when the power of the control device 40 is turned on until when the power of the control device 40 is turned off.

As an example, the display controller 54 is configured or programmed to display the X-ray image display screen 71 and the imaging condition setting screen 72 side by side in the right-left direction of the display 70. The display controller 54 displays the X-ray image display screen 71 and the imaging condition setting screen 72 in this order from the left side of the display 70 when the user looks at the display 70, such that the X-ray image display screen 71 and the imaging condition setting screen 72 do not overlap each other.

As an example, the display controller 54 performs a control to locate a first end 71e of the X-ray image display screen 71 in the right-left direction at a first end 70a of the display 70 in the right-left direction, and performs a control to locate a second end 71f of the X-ray image display screen 71 in the right-left direction at a position based on the pre-selected total number of pixels of the X-ray image display screen 71.

In this embodiment, the display controller 54 performs a control to locate the left end (the first end 71e of the X-ray image display screen 71 in the right-left direction) of the X-ray image display screen 71 in the right-left direction at the left end (the first end 70a of the display 70 in the right-left direction) of the display 70 in the right-left direction, and performs a control to locate the right end (the second end 71f of the X-ray image display screen 71 in the right-left direction) of the X-ray image display screen 71 in the right-left direction at a position based on the pre-selected total number of pixels of the X-ray image display screen 71. That is, for example, the display controller 54 is configured or programmed to display the X-ray image display screen 71 in the range of X coordinates from 0 to 1280 when the display 70 has a total number of pixels of an X coordinate of 1920×a Y coordinate of 1080, and the X-ray image display screen 71 is formed with a total number of pixels of 1280 horizontal pixels×1024 vertical pixels.

As an example, the display controller 54 performs a control to locate a first end 72e of the imaging condition Setting screen 72 in the right-left direction at a second end 70b of the display 70 in the right-left direction, and performs a control to locate a second end 72f of the imaging condition setting screen 72 in the right-left direction at a position based on the pre-selected total number of pixels of the imaging condition setting screen 72.

In this embodiment, the display controller 54 performs a control to locate the right end (the first end 72e of the imaging condition setting screen 72 in the right-left direction) of the imaging condition setting screen 72 in the right-left direction at the right end (the second end 70b of the display 70 in the right-left direction) of the display 70 in the right-left direction, and performs a control to locate the left end (the second end 72f of the imaging condition setting screen 72 in the right-left direction) of the imaging condition setting screen 72 in the right-left direction at a position based on the pre-selected total number of pixels of the imaging condition setting screen 72. That is, for example, the display controller 54 is configured or programmed to display the imaging condition setting screen 72 in the range of X coordinates from 1281 to 1920 when the display 70 has a total number of pixels of an X coordinate of 1920×a Y coordinate of 1080, the X-ray image display screen 71 is formed with a total number of pixels of 1280 horizontal pixels×1024 vertical pixels, and the imaging condition setting screen 72 is formed with a total number of pixels of 640 horizontal pixels×1024 vertical pixels.

In this embodiment, when the X-ray imaging apparatus 100 is installed, the coordinate data 66 of the display position is written and saved in the setting file such that the screen position of the X-ray image display screen 71 has an X coordinate in the range of 0 to 1280. In addition, when the X-ray imaging apparatus 100 is installed, the coordinate data 66 of the display position is written and saved in the setting file such that the screen position of the imaging condition setting screen 72 has an X coordinate in the range of 1281 to 1920. Similarly, for the vertical positions, when the X-ray imaging apparatus 100 is installed, the coordinate data 66 of the display positions is written and saved in the setting file such that the screen positions of the X-ray image display screen 71 and the imaging condition setting screen 72 have a Y coordinate in the range of 29 to 1052. That is, the coordinate data 66 of the display positions is written in the setting file such that the X-ray image display screen 71 and the imaging condition setting screen 72 are located with 28 extra pixels at each of the upper end and lower end of the display 70. When the X-ray image generation software 61 and the imaging condition setting software 62 are started (executed), the display controller 54 reads the setting file and displays the X-ray image display screen 71 and the imaging condition setting screen 72 based on the coordinate data 66 of the display positions.

### Display Processing of X-Ray Image Display Screen and Imaging Condition Setting Screen

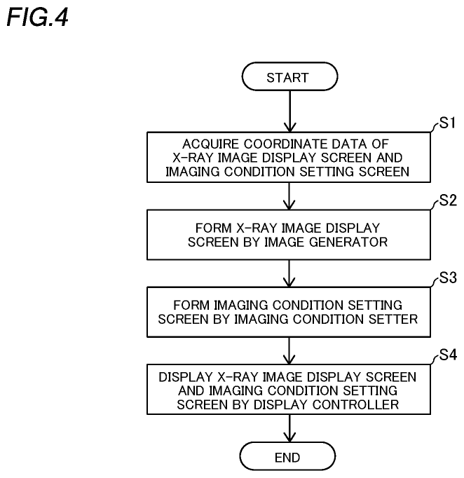

A sequence of display processing of the X-ray image display screen 71 and the imaging condition setting screen 72 according to the embodiment of the present invention is now described with reference to FIG. 4.

In step S1, when the X-ray image generation software 61 and the imaging condition setting software 62 are started (executed), the display controller 54 reads the setting file to acquire the coordinate data 66 of the display positions of the set X-ray image display screen 71 and imaging condition setting screen 72. After that, the process advances to step S2.

In step S2, the image generator 51 uses the X-ray image generation software 61 to form the X-ray image display screen 71. After that, the process advances to step S3.

In step S3, the imaging condition setter 52 uses the imaging condition setting software 62 to form the imaging condition setting screen 72. After that, the process advances to step S4. The order of step S1, step S2, and step S3 may be any one, or the process operations in step S1, step S2, and step S3 may be performed simultaneously.

In step S4, the display controller 54 displays the X-ray image display screen 71 and the imaging condition Setting screen 72 on the display 70. Then, the process is terminated.

### Advantages of This Embodiment

In this embodiment, the following advantages are obtained.

In this embodiment, as described above, the display method in the X-ray imaging apparatus 100 includes a screen formation step of simultaneously and individually executing, on the single control device 40, the X-ray image generation software 61 operable to form the X-ray image based on the detection signal output from the X-ray detector 30 and form the X-ray image display screen 71 and being the product of the first manufacturer, and the imaging condition setting software 62 independent of the X-ray image generation software 61, operable to set the imaging conditions of the X-ray image and form the imaging condition setting screen 72, and being the product of the second manufacturer different from the first manufacturer to form the X-ray image display screen 71 based on the X-ray image generation software 61 and the imaging condition setting screen 72 based on the imaging condition setting software 62, and a screen display step of displaying the X-ray image display screen 71 and the imaging condition setting screen 72 side by side in the different areas of the display 70. Accordingly, the X-ray image generation software 61 and the imaging condition setting software 62, which are made by different manufacturers, are simultaneously and individually executed on the single control device 40 such that the X-ray image display screen 71 and the imaging condition setting screen 72 can be formed and displayed in the different areas of the display 70. Therefore, an environment in which the independently provided X-ray image generation software 61 and imaging condition setting software 62 can be executed simultaneously and individually can be achieved by a flexible combination without restrictions imposed by the manufacturer of the X-ray imaging apparatus 100 and the manufacturer of the X-ray detector 30.

In the embodiment described above, with the following configurations, the following advantages are further obtained.

That is, in this embodiment, as described above, the screen formation step includes forming the X-ray image display screen 71 using the X-ray image generation software 61 such that the total number of pixels of the X-ray image display screen 71 is smaller than the total number of pixels of the entirety of the display 70, and forming the imaging condition setting screen 72 using the imaging condition setting software 62 such that the total number of pixels of the imaging condition setting screen 72 is equal to or less than the number of pixels obtained by subtracting the total number of pixels of the X-ray image display screen 71 from the total number of pixels of the entirety of the display 70. Accordingly, the imaging condition setting screen 72 can be displayed in an area of the single display 70 excluding the area in which the X-ray image display screen 71 is displayed, and thus both the X-ray image display screen 71 based on the X-ray image generation software 61 and the imaging condition setting screen 72 based on the imaging condition setting software 62 can be displayed on the display 70. Therefore, when the X-ray image generation software 61 and the imaging condition setting software 62 are not integrated, the X-ray image display screen 71 and the imaging condition setting screen 72 are displayed on the single display 70 such that the complexity of the configuration of the display 70 and an increase in the number of displays 70 can be reduced or prevented.

In this embodiment, as described above, the screen formation step includes forming the X-ray image display screen 71 using the X-ray image generation software 61 such that the total number of pixels of the X-ray image display screen 71 is a total number of pixels pre-selected from the plurality of types of total numbers of pixels set in advance such that the X-ray image display screen 71 is displayable, and forming the imaging condition setting screen 72 using the imaging condition setting software 62 such that the total number of pixels of the imaging condition setting screen 72 is a total number of pixels pre-selected from the plurality of types of total numbers of pixels set in advance such that the imaging condition setting screen 72 is displayable in an area of the display 70 in which the X-ray image display screen 71 is not displayed. The total number of pixels of the X-ray image display screen 71 is selected from the plurality of types of total numbers of pixels set in advance such that the X-ray image display screen 71 is displayable such that the imaging condition setting screen 72 can be formed using the existing X-ray image generation software 61 directly. In addition, the total number of pixels of the imaging condition setting screen 72 can be selected from the plurality of types of total numbers of pixels based on the selected total number of pixels of the X-ray image display screen 71. For these reasons, when the X-ray image generation software 61 and the imaging condition setting software 62 are not integrated, the X-ray image display screen 71 and the imaging condition setting screen 72 can be easily displayed on the single display 70.

In this embodiment, as described above, the screen display step includes displaying the X-ray image display screen 71 formed using the X-ray image generation software 61 at a fixed position and with a fixed size on the display 70 in a state in which the X-ray image display screen 71 cannot be enlarged or reduced, and displaying the imaging condition setting screen 72 formed using the imaging condition setting software 62 at a fixed position and with a fixed size on the display 70 in a state in which the imaging condition setting screen 72 cannot be enlarged or reduced. The X-ray image display screen 71 and the imaging condition setting screen 72 are screens on which information necessary for X-ray imaging is displayed, and thus it is necessary to display the X-ray image display screen 71 and the imaging condition setting screen 72 on the display 70 during X-ray imaging. The X-ray image display screen 71 and the imaging condition setting screen 72 are displayed at fixed positions and with fixed sizes on the display 70 in a state in which the X-ray image display screen 71 and the imaging condition setting screen 72 cannot be enlarged or reduced such that it is possible to reduce or prevent the possibility that the X-ray image display screen 71 and the imaging condition setting screen 72 become partially or entirely invisible due to changes in the display positions or display sizes of the screens during X-ray imaging.

In this embodiment, as described above, the screen display step includes displaying the X-ray image display screen 71 and the imaging condition setting screen 72 side by side in the right-left direction of the display 70, displaying the first end in the right-left direction of the X-ray image display screen 71 formed using the X-ray image generation software 61 such that the first end 71e of the X-ray image display screen 71 in the right-left direction is located at the first end of the display 70 in the right-left direction and displaying the second end of the X-ray image display screen 71 in the right-left direction such that the second end of the X-ray image display screen 71 in the right-left direction is located at the position based on the pre-selected total number of pixels of the X-ray image display screen 71, and displaying the first end in the right-left direction of the imaging condition setting screen 72 formed using the imaging condition setting software 62 such that the first end of the imaging condition setting screen 72 in the right-left direction is located at the second end of the display 70 in the right-left direction and displaying the second end of the imaging condition setting screen 72 in the right-left direction such that the second end of the imaging condition setting screen 72 in the right-left direction is located at the position based on the pre-selected total number of pixels of the imaging condition setting screen 72. Accordingly, when the X-ray image generation software 61 and the imaging condition setting software 62 are not integrated, the X-ray image display screen 71 and the imaging condition setting screen 72 can be easily displayed on the single display 70.

In this embodiment, as described above, the displayed imaging condition setting screen 72 is operable to enable at least the settings of the tube voltage, the tube current, and the irradiation time. Accordingly, the imaging condition setting screen 72 displayed on the display 70 allows the tube voltage, tube current, and X-ray irradiation time in the X-ray tube (not shown) to be easily set, and allows the tube voltage, tube current, and X-ray irradiation time in the X-ray tube (not shown) to be easily visually recognized.

In this embodiment, as described above, the X-ray imaging apparatus 100 includes the X-ray source 11 to radiate X-rays to the subject, the X-ray detector 30 to detect the X-rays radiated from the X-ray source 11, the display 70, and the single controller 50 configured or programmed to simultaneously and individually execute the X-ray image generation software 61 operable to form the X-ray image based on the detection signal output from the X-ray detector 30 and form the X-ray image display screen 71 and being the product of the first manufacturer, and the imaging condition setting software 62 independent of the X-ray image generation software 61, operable to set the imaging conditions of the X-ray image and form the imaging condition setting screen 72, and being the product of the second manufacturer different from the first manufacturer to form the X-ray image display screen 71 based on the X-ray image generation software 61 and the imaging condition setting screen 72 based on the imaging condition setting software 62, and display the X-ray image display screen 71 based on the X-ray image generation software 61 and the imaging condition setting screen 72 based on the imaging condition setting software 62 side by side in the different areas of the display 70. Accordingly, the X-ray image generation software 61 and the imaging condition Setting software 62, which are made by different manufacturers, are simultaneously and individually executed on the single control device 40 such that the X-ray image display screen 71 and the imaging condition setting screen 72 can be formed and displayed in the different areas of the display 70. Therefore, an environment in which the independently provided X-ray image generation software 61 and imaging condition setting software 62 can be simultaneously and individually executed can be achieved by a flexible combination without restrictions imposed by the manufacturer of the X-ray imaging apparatus 100 and the manufacturer of the X-ray detector 30.

In the embodiment described above, with the following configurations, the following advantages are further obtained.

That is, in this embodiment, as described above, the controller 50 is configured or programmed to perform a control such that the total number of pixels of the X-ray image display screen 71 is smaller than the total number of pixels of the entirety of the display 70 when the X-ray image display screen 71 is formed using the X-ray image generation software 61, and perform a control such that the total number of pixels of the imaging condition setting screen 72 is equal to or less than the number of pixels obtained by subtracting the total number of pixels of the X-ray image display screen 71 from the total number of pixels of the entirety of the display 70 when the imaging condition setting screen 72 is formed using the imaging condition setting software 62. Accordingly, the imaging condition setting screen 72 can be displayed in an area of the single display 70 excluding the area in which the X-ray image display screen 71 is displayed, and thus both the X-ray image display screen 71 based on the X-ray image generation software 61 and the imaging condition Setting screen 72 based on the imaging condition setting software 62 can be displayed on the display 70. Therefore, when the X-ray image generation software 61 and the imaging condition setting software 62 are not integrated, the X-ray image display screen 71 and the imaging condition setting screen 72 are displayed on the single display 70 such that the complexity of the configuration of the display 70 and an increase in the number of displays 70 can be reduced or prevented.

In this embodiment, as described above, the X-ray imaging apparatus 100 includes the X-ray source 11 to radiate X-rays to the subject, the X-ray detector 30 to detect the X-rays radiated from the X-ray source 11, the display 70, and the controller 50 configured or programmed to display the X-ray image display screen 71 based on the X-ray image generation software 61 operable to form the X-ray image based on the detection signal output from the X-ray detector 30 and form the X-ray image display screen 71, and the imaging condition setting screen 72 based on the imaging condition setting software 62 independent of the X-ray image generation software 61 and operable to set the imaging conditions of the X-ray image and form the imaging condition setting screen 72 side by side in the different areas of the display 70, and the controller 50 is configured or programmed to perform a control such that the total number of pixels of the X-ray image display screen 71 is smaller than the total number of pixels of the entirety of the display 70 when the X-ray image display screen 71 is formed using the X-ray image generation software 61, and perform a control such that the total number of pixels of the imaging condition Setting screen 72 is equal to or less than the number of pixels obtained by subtracting the total number of pixels of the X-ray image display screen 71 from the total number of pixels of the entirety of the display 70 when the imaging condition setting screen 72 is formed using the imaging condition setting software 62. The X-ray image display screen 71 based on the X-ray image generation software 61 and the imaging condition setting screen 72 based on the imaging condition setting software 62 are displayed side by side in the different areas of the display 70 such that an environment in which the independently provided X-ray image generation software 61 and imaging condition setting software 62 can be simultaneously and individually executed can be achieved by a flexible combination without restrictions imposed by the manufacturer of the X-ray imaging apparatus 100 and the manufacturer of the X-ray detector 30. In addition, the X-ray image generation software 61 forms the X-ray image display screen 71 such that the total number of pixels of the X-ray image display screen 71 is smaller than the total number of pixels of the entirety of the display 70 in the X-ray imaging apparatus 100, the imaging condition setting software 62 forms the imaging condition setting screen 72 such that the total number of pixels of the imaging condition setting screen 72 is equal to or less than the number of pixels obtained by subtracting the total number of pixels of the X-ray image display screen 71 from the total number of pixels of the entirety of the display 70, and the X-ray image display screen 71 and the imaging condition setting screen 72 are displayed side by side in the different areas of the display 70. Thus, the imaging condition setting screen 72 can be displayed in an area of the single display 70 excluding the area in which the X-ray image display screen 71 is displayed, and thus both the X-ray image display screen 71 based on the X-ray image generation software 61 and the imaging condition setting screen 72 based on the imaging condition setting software 62 can be displayed on the display 70. Therefore, when the X-ray image generation software 61 and the imaging condition setting software 62 are not integrated, the X-ray image display screen 71 and the imaging condition setting screen 72 are displayed on the single display 70 such that the complexity of the configuration of the display 70 and an increase in the number of displays 70 can be reduced or prevented.

### MODIFIED EXAMPLES

The embodiment disclosed this time must be considered as illustrative in all points and not restrictive. The scope of the present invention is not shown by the above description of the embodiment but by the scope of claims for patent, and all modifications (modified examples) within the meaning and scope equivalent to the scope of claims for patent are further included.

For example, while the example in which the X-ray imaging apparatus is configured to image the subject in a posture (lying position) in which the subject is lying has been shown in the aforementioned embodiment, the present invention is not limited to this. For example, the X-ray imaging apparatus may be configured to image a subject in a posture (upright position) in which the subject is standing.

While the example in which the X-ray imaging apparatus is an overhead traveling general X-ray imaging apparatus has been shown in the aforementioned embodiment, the present invention is not limited to this. For example, an X-ray computed tomography (CT) apparatus or a mammographic apparatus may be used.

While the example in which the X-ray image display screen based on the X-ray image generation software and the imaging condition setting screen based on the imaging condition setting software are displayed side by side in the different areas of the display has been shown in the aforementioned embodiment, the present invention is not limited to this. For example, in addition to the X-ray image display screen and the imaging condition setting screen, a screen based on software different from the X-ray image generation software and the imaging condition setting software may be further displayed side by side in a different area of the display.

While the example in which a control is performed such that the total number of pixels of the X-ray image display screen is a total number of pixels pre-selected from the plurality of types of total numbers of pixels set in advance such that the X-ray image display screen is displayable, and a control is performed such that the total number of pixels of the imaging condition setting screen is a total number of pixels pre-selected from the plurality of types of total numbers of pixels set in advance such that the imaging condition setting screen is displayable in an area of the display in which the X-ray image display screen is not displayed has been shown in the aforementioned embodiment, the present invention is not limited to this. For example, the total number of pixels of the X-ray image display screen and the total number of pixels of the imaging condition setting screen may be determined in advance in association with the total number of pixels of the display.

While the example in which the total number of pixels of the X-ray image display screen formed on the display and the total number of pixels of the imaging condition setting screen formed on the display are selected when the X-ray imaging apparatus is installed has been shown in the aforementioned embodiment, the present invention is not limited to this. For example, the total number of pixels of the X-ray image display screen and the total number of pixels of the imaging condition setting screen may be reselectable and changeable during startup of the control device.

While the example in which the X-ray image display screen and the imaging condition setting screen are displayed at fixed positions and with fixed sizes on the display in a state in which the X-ray image display screen and the imaging condition setting screen cannot be enlarged or reduced has been shown in the aforementioned embodiment, the present invention is not limited to this. For example, the X-ray image display screen and the imaging condition setting screen may be displayed on the display in a manner that allows their positions and sizes to be changed while allowing the X-ray image display screen and the imaging condition setting screen to be enlarged or reduced.

While the example in which the X-ray image display screen and the imaging condition setting screen are displayed side by side in the right-left direction of the display has been shown in the aforementioned embodiment, the present invention is not limited to this. For example, the X-ray image display screen and the imaging condition setting screen may be displayed side by side in the upward-downward direction of the display.

While the example in which the X-ray image display screen is displayed on the left side of the display, and the imaging condition setting screen is displayed on the right side of the display has been shown in the aforementioned embodiment, the present invention is not limited to this. For example, the X-ray image display screen may be displayed on the right side of the display, and the imaging condition setting screen may be displayed on the left side of the display.

While the example in which the left end of the X-ray image display screen is located at the left end of the display, and the right end of the imaging condition setting screen is located at the right end of the display has been shown in the aforementioned embodiment, the present invention is not limited to this. For example, the left end of the X-ray image display screen may be located at a position away from the left end of the display, and the right end of the imaging condition setting screen may be located at a position away from the right end of the display.

While the example in which when the imaging conditions of the X-ray image are set by the imaging condition setting software, at least the tube voltage, the tube current, and the X-ray irradiation time are set has been shown in the aforementioned embodiment, the present invention is not limited to this. For example, any one of the tube voltage, the tube current, and the X-ray irradiation time may be set.

While the example in which the display has a total number of pixels of 1920 horizontal pixels×1080 vertical pixels, the X-ray image display screen has a total number of pixels of 1280 horizontal pixels×1024 vertical pixels, and the imaging condition setting screen has a total number of pixels of 640 horizontal pixels×1024 vertical pixels has been shown in the aforementioned embodiment, the present invention is not limited to this. The total number of pixels of the display, the total number of pixels of the X-ray image display screen, and the total number of pixels of the imaging condition setting screen are not particularly limited.

While the example in which the acquired X-ray image data is stored in the storage has been moved has been shown in the aforementioned embodiment, the present invention is not limited to this. For example, the acquired X-ray image data may be stored in a server device (not shown). The server device is, for example, a computer. The server device includes a CPU, a GPU, a ROM, a RAM, etc. as hardware configurations, for example. The server device also includes a non-volatile storage medium. In such a case, the server device is configured to be able to communicate with the control device of the X-ray imaging apparatus.

### ASPECTS

It will be appreciated by those skilled in the art that the exemplary embodiments described above are specific examples of the following aspects.

### Item 1

A display method in an X-ray imaging apparatus, the display method comprising:
a screen formation step of simultaneously and individually executing, on a single control device, X-ray image generation software operable to form an X-ray image based on a detection signal output from an X-ray detector and form an X-ray image display screen, the X-ray image generation software being a product of a first manufacturer, and imaging condition setting software independent of the X-ray image generation software, the imaging condition setting software being operable to set an imaging condition of the X-ray image and form an imaging condition setting screen, the imaging condition setting software being a product of a second manufacturer different from the first manufacturer to form the X-ray image display screen based on the X-ray image generation software and the imaging condition setting screen based on the imaging condition setting software; and
a screen display step of displaying the X-ray image display screen and the imaging condition setting screen side by side in different areas of a display.

### Item 2

The display method in an X-ray imaging apparatus according to item 1, wherein the screen formation step includes forming the X-ray image display screen using the X-ray image generation software such that a total number of pixels of the X-ray image display screen is smaller than a total number of pixels of an entirety of the display, and forming the imaging condition setting screen using the imaging condition setting software such that a total number of pixels of the imaging condition setting screen is equal to or less than a number of pixels obtained by subtracting the total number of pixels of the X-ray image display screen from the total number of pixels of the entirety of the display.

### Item 3

The display method in an X-ray imaging apparatus according to item 2, wherein the screen formation step includes forming the X-ray image display screen using the X-ray image generation software such that the total number of pixels of the X-ray image display screen is the total number of pixels pre-selected from a plurality of types of the total numbers of pixels set in advance such that the X-ray image display screen is displayable, and forming the imaging condition setting screen using the imaging condition setting software such that the total number of pixels of the imaging condition setting screen is the total number of pixels pre-selected from a plurality of types of the total numbers of pixels set in advance such that the imaging condition setting screen is displayable in an area of the display in which the X-ray image display screen is not displayed.

### Item 4

The display method in an X-ray imaging apparatus according to item 3, wherein the screen display step includes displaying the X-ray image display screen formed using the X-ray image generation software at a fixed position and with a fixed size on the display in a state in which the X-ray image display screen cannot be enlarged or reduced, and displaying the imaging condition setting screen formed using the imaging condition setting software at a fixed position and with a fixed size on the display in a state in which the imaging condition setting screen cannot be enlarged or reduced.

### Item 5

The display method in an X-ray imaging apparatus according to item 4, wherein the screen display step includes:
displaying the X-ray image display screen and the imaging condition setting screen side by side in a right-left direction of the display;
displaying a first end in the right-left direction of the X-ray image display screen formed using the X-ray image generation software such that the first end of the X-ray image display screen in the right-left direction is located at a first end of the display in the right-left direction and displaying a second end of the X-ray image display screen in the right-left direction such that the second end of the X-ray image display screen in the right-left direction is located at a position based on the total number of pixels of the X-ray image display screen that has been pre-selected; and
displaying a first end in the right-left direction of the imaging condition setting screen formed using the imaging condition setting software such that the first end of the imaging condition setting screen in the right-left direction is located at a second end of the display in the right-left direction and displaying a second end of the imaging condition setting screen in the right-left direction such that the second end of the imaging condition setting screen in the right-left direction is located at a position based on the total number of pixels of the imaging condition setting screen that has been pre-selected.

### Item 6

The display method in an X-ray imaging apparatus according to item 5, wherein the imaging condition setting screen that has been displayed is operable to enable at least settings of a tube voltage, a tube current, and an X-ray irradiation time.

### Item 7

An X-ray imaging apparatus comprising:
an X-ray source to radiate X-rays to a subject;
an X-ray detector to detect the X-rays radiated from the X-ray source;
a display; and
a single controller configured or programmed to simultaneously and individually execute X-ray image generation software operable to form an X-ray image based on a detection signal output from the X-ray detector and form an X-ray image display screen, the X-ray image generation software being a product of a first manufacturer, and imaging condition setting software independent of the X-ray image generation software, the imaging condition setting software being operable to set an imaging condition of the X-ray image and form an imaging condition setting screen, the imaging condition setting software being a product of a second manufacturer different from the first manufacturer to form the X-ray image display screen based on the X-ray image generation software and the imaging condition setting screen based on the imaging condition setting software, and display the X-ray image display screen based on the X-ray image generation software and the imaging condition setting screen based on the imaging condition setting software side by side in different areas of the display.

### Item 8

The X-ray imaging apparatus according to item 7, wherein the controller is configured or programmed to perform a control such that a total number of pixels of the X-ray image display screen is smaller than a total number of pixels of an entirety of the display when the X-ray image display screen is formed using the X-ray image generation software, and perform a control such that a total number of pixels of the imaging condition setting screen is equal to or less than a number of pixels obtained by subtracting the total number of pixels of the X-ray image display screen from the total number of pixels of the entirety of the display when the imaging condition setting screen is formed using the imaging condition setting software.

### Item 9

An X-ray imaging apparatus comprising:
an X-ray source to radiate X-rays to a subject;
an X-ray detector to detect the X-rays radiated from the X-ray source;
a display; and
a controller configured or programmed to display an X-ray image display screen based on X-ray image generation software operable to form an X-ray image based on a detection signal output from the X-ray detector and form the X-ray image display screen, and an imaging condition setting screen based on imaging condition setting software independent of the X-ray image generation software, the imaging condition setting software being operable to set an imaging condition of the X-ray image and form the imaging condition setting screen side by side in different areas of the display; wherein
the controller is configured or programmed to perform a control such that a total number of pixels of the X-ray image display screen is smaller than a total number of pixels of an entirety of the display when the X-ray image display screen is formed using the X-ray image generation software, and perform a control such that a total number of pixels of the imaging condition setting screen is equal to or less than a number of pixels obtained by subtracting the total number of pixels of the X-ray image display screen from the total number of pixels of the entirety of the display when the imaging condition setting screen is formed using the imaging condition setting software.

### DESCRIPTION OF REFERENCE NUMERALS

1: subject
11: X-ray source
30: X-ray detector
40: control device
50: controller
61: X-ray image generation software
62: imaging condition setting software
70: display
70a: first end of the display in the right-left direction
70b: second end of the display in the right-left direction
71: X-ray image display screen
71e: first end of the X-ray image display screen in the right-left direction
71f: second end of the X-ray image display screen in the right-left direction
72: imaging condition setting screen
72e: first end of the imaging condition setting screen in the right-left direction
72f: second end of the imaging condition setting screen in the right-left direction
100: X-ray imaging apparatus

1. A display method in an X-ray imaging apparatus, the display method comprising:
a screen formation step of simultaneously and individually executing, on a single control device, X-ray image generation software operable to form an X-ray image based on a detection signal output from an X-ray detector and form an X-ray image display screen, the X-ray image generation software being a product of a first manufacturer, and imaging condition setting software independent of the X-ray image generation software, the imaging condition setting software being operable to set an imaging condition of the X-ray image and form an imaging condition setting screen, the imaging condition setting software being a product of a second manufacturer different from the first manufacturer to form the X-ray image display screen based on the X-ray image generation software and the imaging condition setting screen based on the imaging condition setting software; and
a screen display step of displaying the X-ray image display screen and the imaging condition setting screen side by side in different areas of a display.

2. The display method in an X-ray imaging apparatus according to claim 1, wherein the screen formation step includes forming the X-ray image display screen using the X-ray image generation software such that a total number of pixels of the X-ray image display screen is smaller than a total number of pixels of an entirety of the display, and forming the imaging condition setting screen using the imaging condition setting software such that a total number of pixels of the imaging condition setting screen is equal to or less than a number of pixels obtained by subtracting the total number of pixels of the X-ray image display screen from the total number of pixels of the entirety of the display.

3. The display method in an X-ray imaging apparatus according to claim 2, wherein the screen formation step includes forming the X-ray image display screen using the X-ray image generation software such that the total number of pixels of the X-ray image display screen is a total number of pixels pre-selected from a plurality of types of total numbers of pixels set in advance such that the X-ray image display screen is displayable, and forming the imaging condition setting screen using the imaging condition setting software such that the total number of pixels of the imaging condition setting screen is a total number of pixels pre-selected from a plurality of types of total numbers of pixels set in advance such that the imaging condition setting screen is displayable in an area of the display in which the X-ray image display screen is not displayed.

4. The display method in an X-ray imaging apparatus according to claim 3, wherein the screen display step includes displaying the X-ray image display screen formed using the X-ray image generation software at a fixed position and with a fixed size on the display in a state in which the X-ray image display screen cannot be enlarged or reduced, and displaying the imaging condition setting screen formed using the imaging condition setting software at a fixed position and with a fixed size on the display in a state in which the imaging condition setting screen cannot be enlarged or reduced.

5. The display method in an X-ray imaging apparatus according to claim 4, wherein the screen display step includes:
displaying the X-ray image display screen and the imaging condition setting screen side by side in a right-left direction of the display;
displaying a first end in the right-left direction of the X-ray image display screen formed using the X-ray image generation software such that the first end of the X-ray image display screen in the right-left direction is located at a first end of the display in the right-left direction and displaying a second end of the X-ray image display screen in the right-left direction such that the second end of the X-ray image display screen in the right-left direction is located at a position based on the total number of pixels of the X-ray image display screen that has been pre-selected; and
displaying a first end in the right-left direction of the imaging condition setting screen formed using the imaging condition setting software such that the first end of the imaging condition setting screen in the right-left direction is located at a second end of the display in the right-left direction and displaying a second end of the imaging condition setting screen in the right-left direction such that the second end of the imaging condition setting screen in the right-left direction is located at a position based on the total number of pixels of the imaging condition setting screen that has been pre-selected.

6. The display method in an X-ray imaging apparatus according to claim 5, wherein the imaging condition setting screen that has been displayed is operable to enable at least settings of a tube voltage, a tube current, and an X-ray irradiation time.

7. An X-ray imaging apparatus comprising:
an X-ray source to radiate X-rays to a subject;
an X-ray detector to detect the X-rays radiated from the X-ray source;
a display; and
a single controller configured or programmed to simultaneously and individually execute X-ray image generation software operable to form an X-ray image based on a detection signal output from the X-ray detector and form an X-ray image display screen, the X-ray image generation software being a product of a first manufacturer, and imaging condition setting software independent of the X-ray image generation software, the imaging condition setting software being operable to set an imaging condition of the X-ray image and form an imaging condition setting screen, the imaging condition setting software being a product of a second manufacturer different from the first manufacturer to form the X-ray image display screen based on the X-ray image generation software and the imaging condition setting screen based on the imaging condition setting software, and display the X-ray image display screen based on the X-ray image generation software and the imaging condition setting screen based on the imaging condition setting software side by side in different areas of the display.

8. The X-ray imaging apparatus according to claim 7, wherein the controller is configured or programmed to perform a control such that a total number of pixels of the X-ray image display screen is smaller than a total number of pixels of an entirety of the display when the X-ray image display screen is formed using the X-ray image generation software, and perform a control such that a total number of pixels of the imaging condition setting screen is equal to or less than a number of pixels obtained by subtracting the total number of pixels of the X-ray image display screen from the total number of pixels of the entirety of the display when the imaging condition setting screen is formed using the imaging condition setting software.

9. An X-ray imaging apparatus comprising:
an X-ray source to radiate X-rays to a subject;
an X-ray detector to detect the X-rays radiated from the X-ray source;
a display;
a controller configured or programmed to display an X-ray image display screen based on X-ray image generation software operable to form an X-ray image based on a detection signal output from the X-ray detector and form the X-ray image display screen, and an imaging condition setting screen based on imaging condition setting software independent of the X-ray image generation software, the imaging condition setting software being operable to set an imaging condition of the X-ray image and form the imaging condition setting screen side by side in different areas of the display; and
a storage to store a plurality of types of total numbers of pixels of each of the X-ray image display screen and the imaging condition setting screen,
wherein the controller is configured or programmed to:
set a total number of pixels selected from the plurality of types of total numbers of pixels stored in the storage as a total number of pixels of a corresponding X-ray image display screen or imaging condition setting screen, and
perform a control such that a total number of pixels of the X-ray image display screen is smaller than a total number of pixels of an entirety of the display, using a set total number of pixels when the X-ray image display screen is formed using the X-ray image generation software, and perform a control such that a total number of pixels of the imaging condition setting screen is equal to or less than a number of pixels obtained by subtracting the total number of pixels of the X-ray image display screen from the total number of pixels of the entirety of the display, using a set total number of pixels when the imaging condition setting screen is formed using the imaging condition setting software.

10. The X-ray imaging apparatus according to claim 9, wherein the controller is configured or programmed to display the X-ray image display screen formed using the X-ray image generation software at a fixed position and with a fixed size on the display in a state in which the X-ray image display screen cannot be enlarged or reduced, and display the imaging condition setting screen formed using the imaging condition setting software at a fixed position and with a fixed size on the display in a state in which the imaging condition setting screen cannot be enlarged or reduced.

11. The X-ray imaging apparatus according to claim 10, wherein the controller is configured or programmed to:
display the X-ray image display screen and the imaging condition setting screen side by side in a right-left direction of the display,
display a first end in the right-left direction of the X-ray image display screen formed using the X-ray image generation software such that the first end of the X-ray image display screen in the right-left direction is located at a first end of the display in the right-left direction and display a second end of the X-ray image display screen in the right-left direction such that the second end of the X-ray image display screen in the right-left direction is located at a position based on the total number of pixels of the X-ray image display screen that has been pre-selected, and
display a first end in the right-left direction of the imaging condition setting screen formed using the imaging condition setting software such that the first end of the imaging condition setting screen in the right-left direction is located at a second end of the display in the right-left direction and display a second end of the imaging condition setting screen in the right-left direction such that the second end of the imaging condition setting screen in the right-left direction is located at a position based on the total number of pixels of the imaging condition setting screen that has been pre-selected.
